(19) United States
(12) Patent Application Publication
(10) Pub. No.: US 2024/0077526 A1
(43) Pub. Date: Mar. 7, 2024

(54) HARDWARE NOISE FILTERING

(71) Applicant: Oura Health Oy, Oulu (FI)

(72) Inventors: Mika Petteri Kangas, Oulu (FI); Jukka- Tapani Mäkinen, Oulu (FI); Jaakko Tapio Vartiainen, Oulu (FI); Olli Petteri Heikkinen, Oulu (FI); Kirsi Marja Maansaari, Oulu (FI)

(21) Appl. No.: 17/903,661

(22) Filed: Sep. 6, 2022

(51) Int. Cl. G01R 29/26; G01R 31/00

(52) U.S. Cl. CPC G01R 29/26; G01R 31/003

(57) ABSTRACT

Methods, systems, and devices for hardware noise filtering are described. A wearable device may emit light from a set of light emitting elements (e.g., light emitting diodes (LEDs)) based on a known input signal. The wearable device may measure an output signal at a set of photodetectors. The output signal may be generated by passing the light emitted from the set of light emitting elements into a material in contact with the wearable device. The wearable device may compare the known input signal to the output signal and may determine a hardware noise component of the output signal based on a known environmental noise component of the output signal and the comparison. The wearable device may store the hardware noise component for filtering hardware noise from sensor measurements.

### FIELD OF TECHNOLOGY

The following relates to wearable devices and data processing, including hardware noise filtering.

### BACKGROUND

Some wearable devices may be configured to collect data from users associated with a physiological phenomenon, motion artifact, or both, such as a heart rate, oxygen saturation level, blood pressure, or the like. However, there may be variability between sensors in wearable devices, which may cause hardware noise. As such, there are technical challenges with calibrating sensors to account for hardware noise of wearable devices.

### BRIEF DESCRIPTION OF THE DRAWINGS

FIG. 1 illustrates an example of a system that supports hardware noise filtering in accordance with aspects of the present disclosure.

FIG. 2 illustrates an example of a system that supports hardware noise filtering in accordance with aspects of the present disclosure.

FIGS. 3 and 4 illustrate examples of calibration diagrams that support hardware noise filtering in accordance with aspects of the present disclosure.

FIG. 5 shows a block diagram of an apparatus that supports hardware noise filtering in accordance with aspects of the present disclosure.

FIG. 6 shows a block diagram of a wearable device manager that supports hardware noise filtering in accordance with aspects of the present disclosure.

FIG. 7 shows a diagram of a system including a device that supports hardware noise filtering in accordance with aspects of the present disclosure.

FIGS. 8 through 10 show flowcharts illustrating methods that support hardware noise filtering in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, pressure data, heart rate data, and the like. In order to efficiently and accurately track physiological data, a wearable device may be configured to collect data continuously while the user wears the device.

In some cases, measurements from different sensors of a wearable device may vary. For example, physiological data, mobility measurements, or both from a wearable device may be subject to hardware noise from the variability in the different sensors. The components, or elements, of the sensors in the wearable device may produce hardware noise, which is an interference value in an output signal attributed to the hardware of the wearable device. In some examples, there may be different types of noise in addition to hardware noise, such as an environmental noise due to the environment of the user (e.g., an external light that interferes with a signal from light emitting elements of the wearable device). The different types of noise may cause inconsistencies in processing of the data, mobility measurements, or both. Thus, one or more wearable device sensors of a wearable device (e.g., a ring, watch, necklace, earring, or any other wearable device placed on a human body part) may be calibrated to accurately collect physiological data. However, the calibration process may not isolate the hardware noise from the other types of noise, and therefore may be overlooked, causing the sensors to be improperly calibrated. A lack of proper calibration may lead to inaccurate or misleading physiological data or a negative experience for the user of the wearable device.

Accordingly, techniques described herein are directed to devices, methods, and systems for calibrating one or more sensors of a wearable device by filtering out a hardware noise component of an output signal. In some examples, a wearable device may isolate the hardware noise component of the output signal by using a known environmental noise component and a known input signal. For example, to obtain the known environmental noise component, a user calibrating the wearable device sensors may use a device made to represent a human appendage, such as a body component or an artificial digit. In some cases, a finger phantom, which may also be referred to as an artificial digit, may be configured to mimic human tissue and may be in the shape of a human finger or any body part (e.g., a human appendage, a human chest, a human neck, a human head, or any other body part). For example, the artificial digit may be formed from one or more materials with optical, thermal, or mechanical properties that mimic or represent the properties of human tissue.

The wearable device may compare a known input signal to a set of light emitting elements (e.g., light emitting diodes (LEDs), or any other light emitting sensor component) to a measured output signal at a set of photodetectors. The wearable device may determine the hardware noise component by performing an operation to eliminate the known environmental noise component from the comparison of the known input signal and the measured output signal. The operation may include subtracting the known environmental noise component, applying an algorithm to eliminate the known environmental noise component, or otherwise filtering out the known environmental noise component. The wearable device may store the hardware noise component (e.g., at a cloud server, at a user device, at the wearable device, or any other memory the wearable device has access to). In some cases, the wearable device may use the stored hardware noise component to filter out hardware noise from measurements of a physiological phenomenon of the wearer of the wearable device.

Aspects of the disclosure are initially described in the context of systems supporting physiological data collection from users via wearable devices. Additional aspects of the disclosure are described in the context of calibration diagrams. Aspects of the disclosure are further illustrated by and described with reference to apparatus diagrams, system diagrams, and flowcharts that relate to hardware noise filtering.

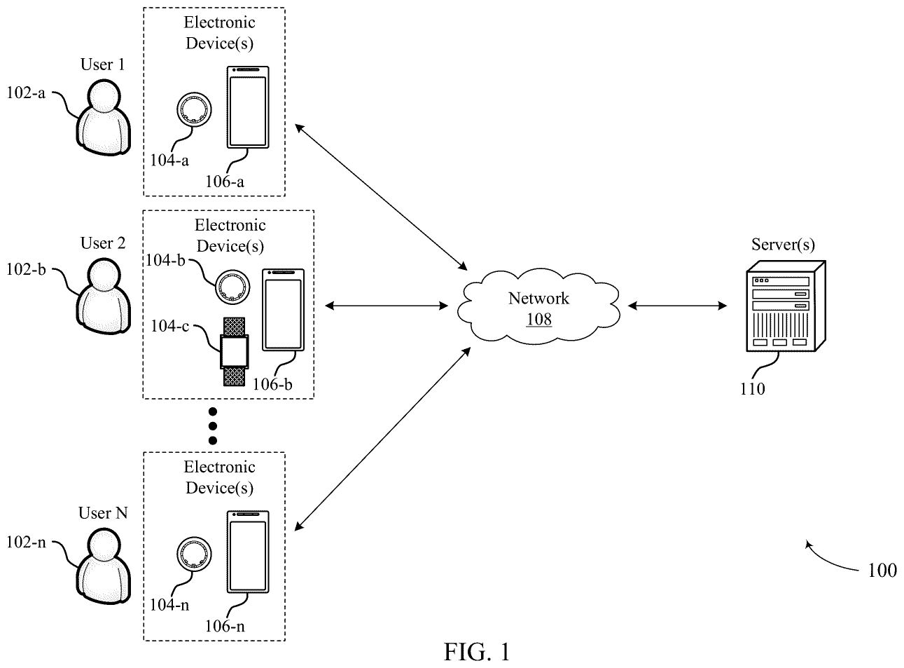

FIG. 1 illustrates an example of a system 100 that supports hardware noise filtering in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via graphical user interfaces (GUIs)) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) that emit light on the palm-side of a user's finger to collect physiological data based on arterial blood flow within the user's finger. In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

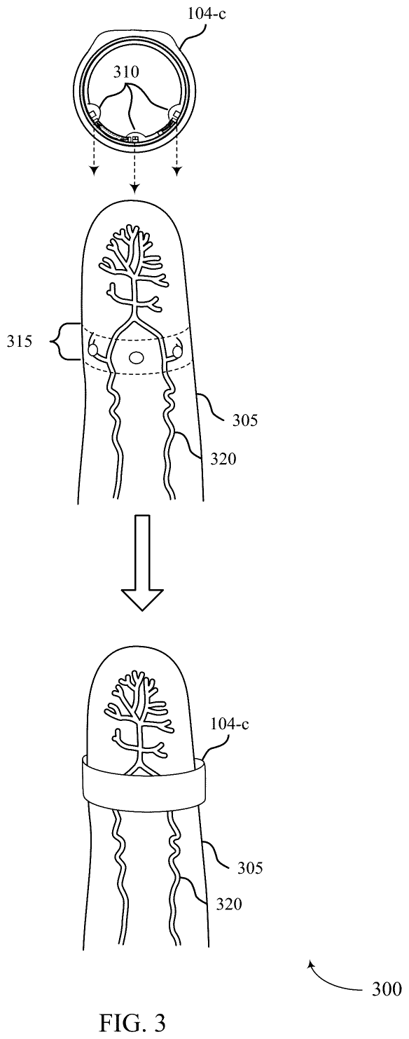

In some aspects, the respective devices of the system 100 may support techniques for using a known environmental noise component to filter a hardware noise component from one or more measurements of a physiological phenomenon of a wearer (e.g., a user 102) of a wearable device 104. For example, the wearable device 104 may be environmentally controlled calibration setup, such as being placed on an artificial digit, or phantom finger, made of a polymer material that mimics optical properties, thermal properties, mechanical properties, or any combination thereof of human tissue (e.g., a human finger). Additionally, or alternatively, the wearable device 104 may determine the known environmental noise component using one or more sensor measurements from the wearable device 104. The wearable device 104 may use a known input signal, a measured output signal, and the environmental noise component to derive a hardware noise component of the output signal, which is described in further detail with respect to FIG. 3.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

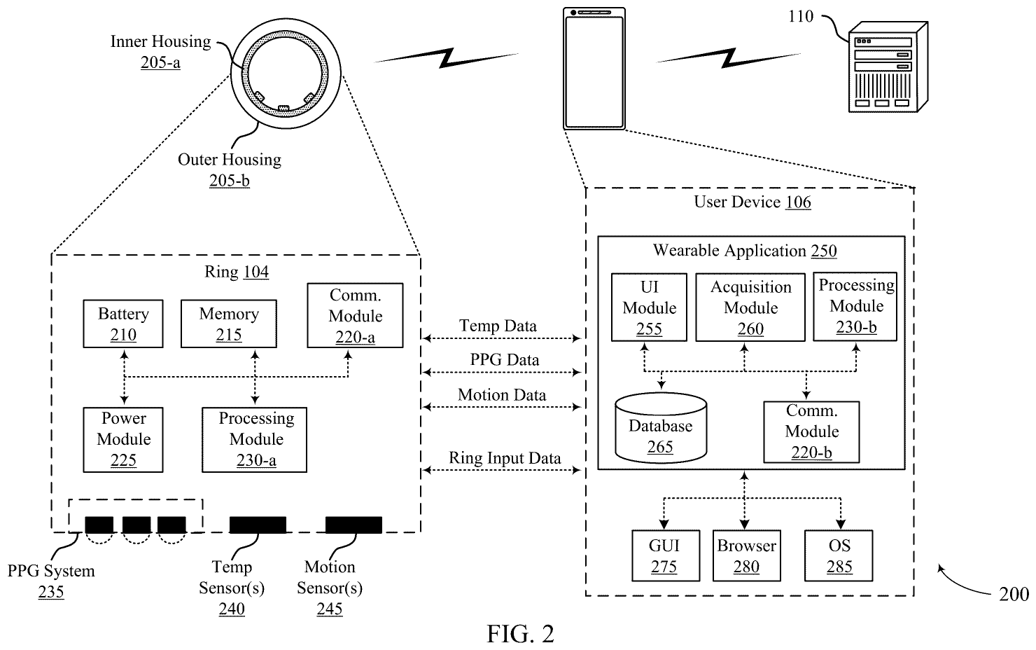

FIG. 2 illustrates an example of a system 200 that supports hardware noise filtering in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG LEDs. In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for calibrating a wearable device using one or more measurements from a set of light emitting elements and a set of photodetectors of a wearable device. The measurements may be referred to as an output signal, and may be measured based on a known input signal to the light emitting elements. In some cases, the light emitting elements may be examples of LEDs, as described with reference to the PPG system 235. The wearable device may use a known environmental noise component, the known input signal, and the measured output signal to derive, or otherwise obtain, a hardware noise component of the output signal. For example, the wearable device may compare the output signal to the known input signal, and may subtract, or otherwise eliminate, the known environmental noise component from the output signal. The remainder may be the hardware noise component of the output signal, which may be caused by the one or more sensors (e.g., sensors of the PPG system 235) of the wearable device. The wearable device may store the hardware noise component of the output signal at the wearable device, at the user device 106, or at a server 110 (e.g., a cloud server). The wearable device may use the hardware noise component of the output signal to filter hardware noise from one or more measurements by the PPG system 235 when collecting PPG data, motion data, temperature data, or the like.

FIG. 3 illustrates an example of a calibration diagram 300 that supports hardware noise filtering in accordance with aspects of the present disclosure. The calibration diagram 300 may implement, or be implemented by, aspects of the system 100, the system 200, or both. The calibration diagram 300 may illustrate the coupling of a wearable device 104-c with a human appendage or an artificial human appendage, which may be referred to as a digit 305. The wearable device 104-c may be placed on a digit 305 for measurements and sensor calibration. Although the wearable devices are illustrated as rings in FIG. 3, they may be any example of a wearable device (e.g., a watch, a necklace, and the like). Similarly, although the digit 305 is illustrated as a finger in FIG. 3, the digit 305 may represent any human body part for any example of a wearable device (e.g., a wrist for a watch, a neck for a necklace, and the like).

In some examples, the wearable device 104-c may have one or more wearable device sensors 310, which may be examples of the sensors in the PPG system, the motion sensors, the temperature sensors, or the like as described with reference to FIG. 2. The wearable device sensors 310 may include LEDs, pressure sensors, thermal sensors, or the like, for detecting optical, thermal, and mechanical properties. There may be any number of wearable device sensors 310 at any locations of the wearable device 104-c. The wearable device sensors 310 may vary in size and distance from each other. A coupling site 315 may be one or more coupling sites for one or more wearable devices and may be located anywhere on the digit 305. For example, the wearable device 104-c may include one or more light emitting elements, which may be examples of LEDs, micro LEDs, mini LEDs, laser diodes (LDs), or any other light emitting component, and one or more photodetectors. The light emitting elements and the photodetectors may make up a sensor pair. For example, each sensor 310 may include a light emitting element and a photodetector.

In some cases, the wearable device 104-c may activate the sensors 310 to perform one or more measurements, such as measurements of a physiological phenomenon of a user. Some examples of the physiological phenomenon may include a heart rate of the user, an oxygen saturation level (e.g., blood oxygen saturation (SpO2)), a blood pressure, or any combination thereof. Prior to performing the one or more measurements, the wearable device 104-c may calibrate the sensors 310. For example, the wearable device 104-c may calibrate the one or more wearable device sensors 310 to account for different types of noise, such as noise related to variability in a user's physiological features, noise from the environment (e.g., an ambient light noise, motion artifact noise, physiological phenomenon noise, or the like), and hardware noise. However, the wearable device 104-c may not be able to distinguish the different types of noise from each other. That is, the wearable device 104-c may detect a single noise value, which may cause errors in the calibration due to variation in hardware noise values between wearable devices and between individual wearable device sensors 310.

In some examples, to detect the hardware noise component of a measurement, the wearable device 104-c may determine a known environmental noise component of the measurement. For example, a user calibrating the wearable device 104-c may couple the wearable device 104-c with an artificial digit, which may include one or more elements, such as channels 320 representative of human veins (e.g., arteries and capillaries) or electrochromic sheets, liquid crystal layers, a spatial light modulator, or any combination thereof that may adjust light absorption properties of the polymer material, light transmission properties of the polymer material, or the like. The artificial digit may also include one or more artifacts representative of human skin, human ligaments, human bone, or any combination thereof.

In some other examples, the user calibrating the wearable device 104-c may receive instructions via a user device (e.g., a GUI of the user device) to calibrate the wearable device 104-c to obtain a known environmental noise. That is, the user may be instructed to place the wearable device 104-c out of the way of ambient light, at an orientation or position on a body part, at a time of day, for a physical activity (e.g., lying down), may be instructed to refrain from performing physical activity, or the like to control the environment of the user. The wearable device 104-c may perform one or more sensor measurements on one or more channels 320 (e.g., human veins) with the controlled, or static, environment of the user to obtain a known environmental noise. For example, the wearable device 104-c may perform measurements throughout a duration (e.g., a night) according to a set of movements, and may compare how the values of the measurements change to obtain the environmental noise. Thus, the wearable device 104-c may obtain a known environmental noise from the digit 305, via the artificial digit or the body part in a controlled environment.

In some examples, the wearable device sensors 310 may output a number of signal paths, such as 18 signal paths. For example, one or more light emitting elements of the wearable device 104-c may emit light according to a known input signal. The input signal may include the number of signal paths to account for the number of wearable device sensors 310. A set of photodetectors may measure an output signal generated by passing the light emitted from the light emitting elements into the digit 305 (e.g., a material in contact with the wearable device 104-c). The wearable device 104-c may compare the known input signal to the output signal to obtain a noise estimate. The wearable device 104-c may determine a hardware component of the noise estimate (e.g., of the output signal) using the known environmental noise component and the noise estimate. For example, the wearable device 104-c may apply a post-processing algorithm to extract the hardware noise component and to build a noise model for the hardware noise. In some cases, the post-processing algorithm may include subtracting the known environmental noise from the noise estimate to obtain a remainder of noise, which may be attributed to hardware noise.

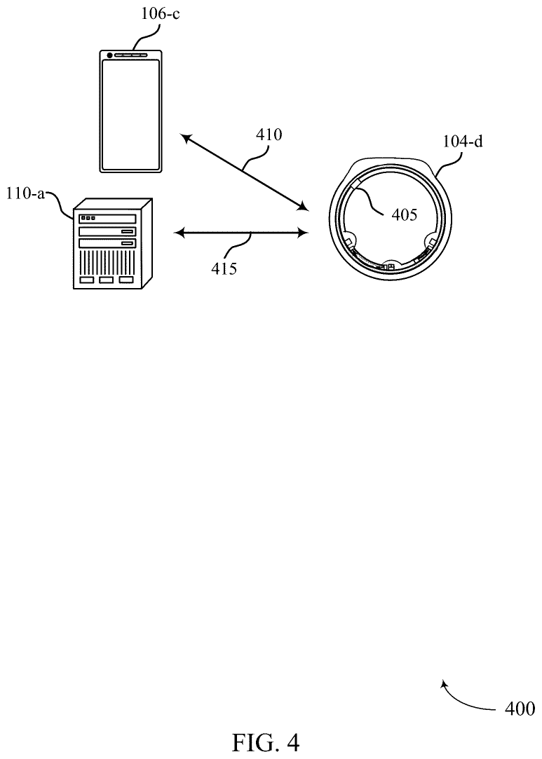

In some cases, the wearable device 104-c may store the hardware noise component, or the noise model for the hardware noise, which is described in further detail with respect to FIG. 4. The wearable device 104-c may use the stored hardware noise component to filter out the hardware noise as a pre-processing step, or element, before feeding a signal to one or more algorithms for post-processing. For example, the wearable device 104-c may activate the wearable device sensors 310 to collect measurements related to a physiological phenomenon of a wearer of the wearable device 104-c (e.g., heart rate values, IBI values, HRV values, respiratory rate values, SpO2, blood pressure, or the like). The wearable device 104-c may use the stored hardware noise to filter out the hardware noise component of the collected measurements.

In some examples, the wearable device 104-c may perform parameter tuning in relation to the hardware noise component of the output signal. For example, the wearable device 104-c may measure and compare hardware noise for a parameter matrix, which may include a set of parameters for the wearable device sensors 310 (e.g., a set of parameters that controls the input signal). The set of parameters may define the input signal to the light emitting elements, such as a power of the light emitting elements. The wearable device 104-c may measure an impact of each parameter on the output signal and, specifically, the hardware noise component. For example, the wearable device 104-c may perform measurements using the set of light emitting elements and the set of photodetectors. Each measurement may relate to a different parameter of a parameter configuration (e.g., the parameter matrix). The wearable device 104-c may determine hardware noise values for each measurement parameter configuration.

The wearable device 104-c may define a minimum and maximum set of parameters (e.g., usable parameters) based on comparing the hardware noise component for each parameter to a threshold hardware noise component. For example, the wearable device 104-c may sort hardware noise values for each measurement configuration to obtain a measurement parameter configuration range that satisfies a measurement accuracy value. The wearable device 104-c may store the measurement parameter configuration range for later use. In some cases, the minimum and maximum set of parameters may be based on a use case (e.g., an SpO2 measurement, a dihydrorhodamine (DHR) measurement, or the like), a type of wearable device, a user, a portion of an input signal, or any other factor. The parameter tuning may improve component selection (e.g., selection of the sensors 310, or other components of the wearable device 104-c), may provide factory tuning parameters when the wearable device 104-c is manufactured, reduce or mitigate component variance and corresponding impact of hardware noise on the wearable device 104-c, and the like.

FIG. 4 illustrates an example of a calibration diagram 400 that supports hardware noise filtering in accordance with aspects of the present disclosure. The calibration diagram 400 may implement, or be implemented by, aspects of the system 100, the system 200, the calibration diagram 300, or any combination thereof. The calibration diagram 400 may illustrate an example of a wearable device 104-d storing a hardware noise component of an output signal for future use. Although the wearable device 104-d is illustrated as a ring in FIG. 4, the wearable device 104-d may be any example of a wearable device (e.g., a watch, a necklace, and the like).

In some cases, the wearable device 104-d may use one or more light emitting elements and photodetectors to obtain a hardware noise component of an output signal based on a known input signal and a known environmental noise, as described with reference to FIG. 2. In some examples, the wearable device 104-d may store the hardware noise component, or the noise model for the hardware noise. For example, the wearable device 104-d may store the hardware noise component or a noise model for the hardware noise at local memory 405 of the wearable device 104-d. The wearable device 104-d may access the local memory 405 to obtain the stored hardware noise component or the noise model when filtering out a noise component of an output signal. Thus, the filtering of the hardware noise component may occur at the processor of the wearable device 104-d.

In some other examples, the wearable device 104-d may be in contact with a user device 106-c via a communication link 410, a server 110-a via a communication link 415, or both. The wearable device 104-d may obtain one or more hardware noise components of output signals, and may store the hardware noise component or the noise model for the hardware noise at the user device 106-c, at the server 110-a, or both. The wearable device 104-d may access the user device 106-c, the server 110-a, or both via the communication link 410 and the communication link 415, respectively, when filtering out the hardware noise component of an output signal. In some cases, the server 110-a may be an example of a cloud server.

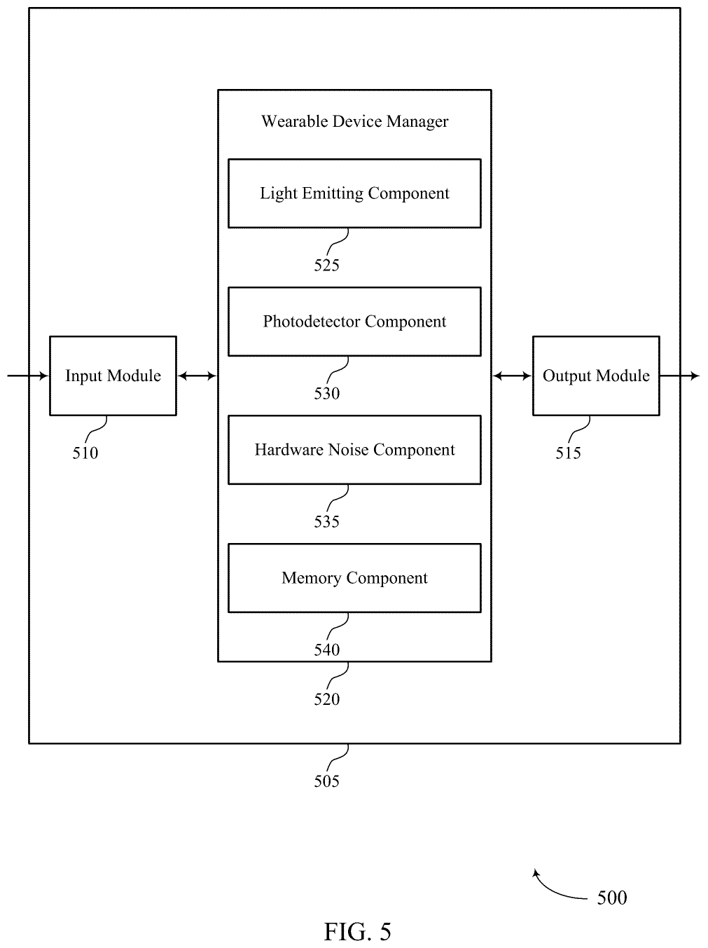

FIG. 5 shows a block diagram 500 of a device 505 that supports hardware noise filtering in accordance with aspects of the present disclosure. The device 505 may include an input module 510, an output module 515, and a wearable device manager 520. The device 505 may also include a processor. Each of these components may be in communication with one another (e.g., via one or more buses).

For example, the wearable device manager 520 may include a light emitting component 525, a photodetector component 530, a hardware noise component 535, a memory component 540, or any combination thereof. In some examples, the wearable device manager 520, or various components thereof, may be configured to perform various operations (e.g., receiving, monitoring, transmitting) using or otherwise in cooperation with the input module 510, the output module 515, or both. For example, the wearable device manager 520 may receive information from the input module 510, send information to the output module 515, or be integrated in combination with the input module 510, the output module 515, or both to receive information, transmit information, or perform various other operations as described herein.

The wearable device manager 520 may support hardware noise filtering for a wearable device in accordance with examples as disclosed herein. The light emitting component 525 may be configured as or otherwise support a means for emitting light from a set of light emitting elements of the wearable device based at least in part on a known input signal to the set of light emitting elements. The photodetector component 530 may be configured as or otherwise support a means for measuring, at a set of photodetectors of the wearable device, an output signal, wherein the output signal is generated by passing the light emitted from the set of light emitting elements into a material in contact with the wearable device. The hardware noise component 535 may be configured as or otherwise support a means for determining a hardware noise component of the output signal based at least in part on a known environmental noise component of the output signal and a comparison of the known input signal to the output signal. The memory component 540 may be configured as or otherwise support a means for storing the hardware noise component.

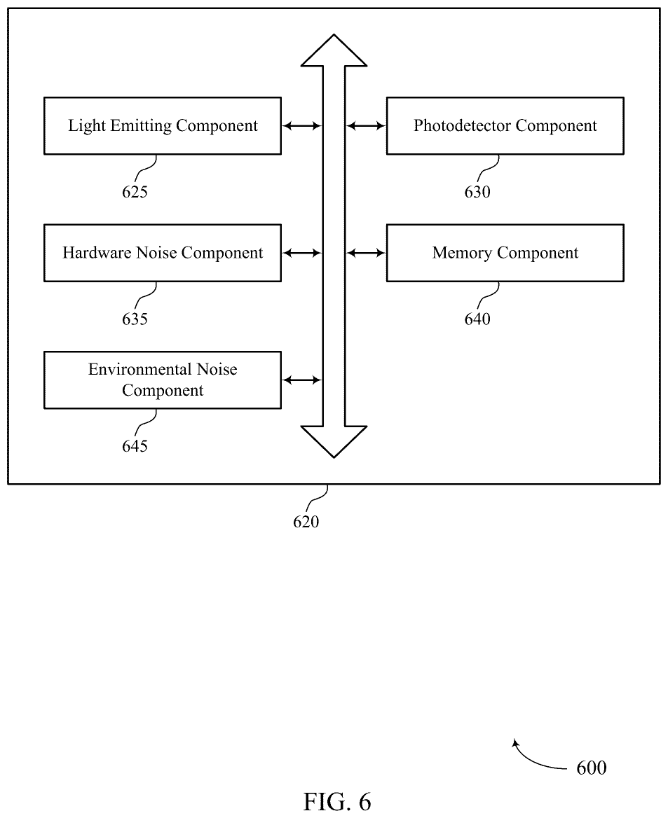

FIG. 6 shows a block diagram 600 of a wearable device manager 620 that supports hardware noise filtering in accordance with aspects of the present disclosure. The wearable device manager 620 may be an example of aspects of a wearable device manager or a wearable device manager 520, or both, as described herein. The wearable device manager 620, or various components thereof, may be an example of means for performing various aspects of hardware noise filtering as described herein. For example, the wearable device manager 620 may include a light emitting component 625, a photodetector component 630, a hardware noise component 635, a memory component 640, an environmental noise component 645, or any combination thereof. Each of these components may communicate, directly or indirectly, with one another (e.g., via one or more buses).

The wearable device manager 620 may support hardware noise filtering for a wearable device in accordance with examples as disclosed herein. The light emitting component 625 may be configured as or otherwise support a means for emitting light from a set of light emitting elements of the wearable device based at least in part on a known input signal to the set of light emitting elements. The photodetector component 630 may be configured as or otherwise support a means for measuring, at a set of photodetectors of the wearable device, an output signal, wherein the output signal is generated by passing the light emitted from the set of light emitting elements into a material in contact with the wearable device. The hardware noise component 635 may be configured as or otherwise support a means for determining a hardware noise component of the output signal based at least in part on a known environmental noise component of the output signal and a comparison of the known input signal to the output signal. The memory component 640 may be configured as or otherwise support a means for storing the hardware noise component.

In some examples, to support determining the hardware noise component, the environmental noise component 645 may be configured as or otherwise support a means for filtering out the known environmental noise component from the output signal.

In some examples, the known environmental noise component comprises ambient light noise, motion artifact noise, physiological phenomenon noise, or a combination thereof.

In some examples, a physiological phenomenon corresponding to the physiological phenomenon noise comprises a heart rate, an oxygen saturation level, a blood pressure, or a combination thereof.

In some examples, the environmental noise component 645 may be configured as or otherwise support a means for determining the known environmental noise component based at least in part on one or more sensor measurements from the wearable device.

In some examples, the environmental noise component 645 may be configured as or otherwise support a means for determining the known environmental noise component based at least in part on an environmentally controlled calibration setup, wherein the material in contact with the wearable device comprises an artificial material configured to mimic an optical property of human tissue.

In some examples, the light emitting component 625 may be configured as or otherwise support a means for activating, after storing the hardware noise component, the wearable device to collect a plurality of measurements of a physiological phenomenon of a wearer of the wearable device. In some examples, the hardware noise component 635 may be configured as or otherwise support a means for filtering out the hardware noise component from the plurality of measurements.

In some examples, the light emitting component 625 may be configured as or otherwise support a means for performing a plurality of measurements using the set of light emitting elements and the set of photodetectors, wherein each measurement of the plurality of measurements corresponds to a different measurement parameter configuration. In some examples, the hardware noise component 635 may be configured as or otherwise support a means for determining one or more hardware noise values for each measurement parameter configuration.

In some examples, the hardware noise component 635 may be configured as or otherwise support a means for sorting the one or more hardware noise values for each measurement configuration to obtain a measurement parameter configuration range satisfying a threshold measurement accuracy value. In some examples, the memory component 640 may be configured as or otherwise support a means for storing the measurement parameter configuration range.

In some examples, the output signal corresponds to a plurality of signal paths associated with the set of light emitting elements and the set of photodetectors.

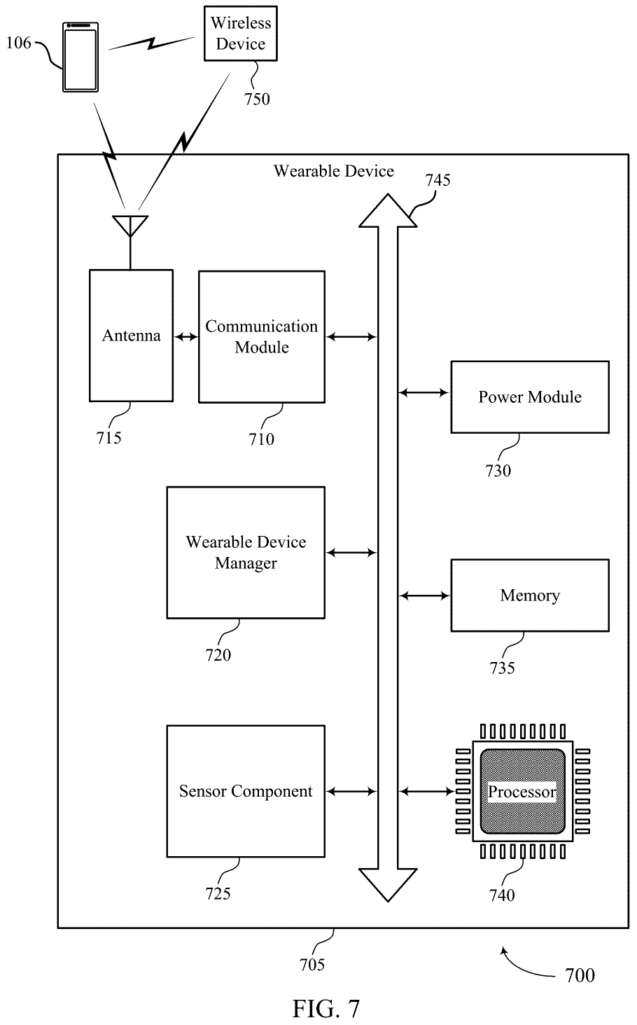

FIG. 7 shows a diagram of a system 700 including a device 705 that supports hardware noise filtering in accordance with aspects of the present disclosure. The device 705 may be an example of or include the components of a device 505 as described herein. The device 705 may include an example of a wearable device 104, as described previously herein. The device 705 may include components for bi-directional communications including components for transmitting and receiving communications with a user device 106 and a server 110, such as a wearable device manager 720, a communication module 710, an antenna 715, a sensor component 725, a power module 730, a memory 735, a processor 740, and a wireless device 750. These components may be in electronic communication or otherwise coupled (e.g., operatively, communicatively, functionally, electronically, electrically) via one or more buses (e.g., a bus 745).

The wearable device manager 720 may support hardware noise filtering for a wearable device in accordance with examples as disclosed herein. For example, the wearable device manager 720 may be configured as or otherwise support a means for emitting light from a set of light emitting elements of the wearable device based at least in part on a known input signal to the set of light emitting elements. The wearable device manager 720 may be configured as or otherwise support a means for measuring, at a set of photodetectors of the wearable device, an output signal, wherein the output signal is generated by passing the light emitted from the set of light emitting elements into a material in contact with the wearable device. The wearable device manager 720 may be configured as or otherwise support a means for determining a hardware noise component of the output signal based at least in part on a known environmental noise component of the output signal and a comparison of the known input signal to the output signal. The wearable device manager 720 may be configured as or otherwise support a means for storing the hardware noise component.

By including or configuring the wearable device manager 720 in accordance with examples as described herein, the device 705 may support techniques for a wearable device to obtain a hardware noise component of an output signal, which may provide for improved accuracy related to measurement calibration.

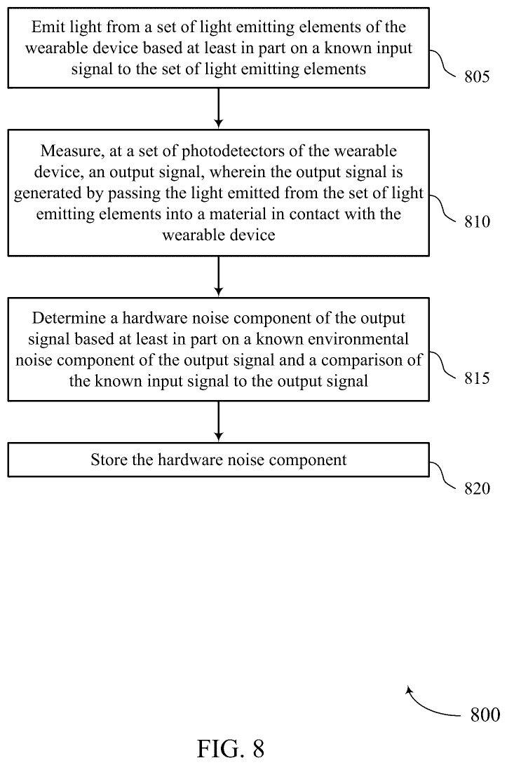

FIG. 8 shows a flowchart illustrating a method 800 that supports hardware noise filtering in accordance with aspects of the present disclosure. The operations of the method 800 may be implemented by a wearable device or its components as described herein. For example, the operations of the method 800 may be performed by a wearable device as described with reference to FIGS. 1 through 7. In some examples, a wearable device may execute a set of instructions to control the functional elements of the wearable device to perform the described functions. Additionally, or alternatively, the wearable device may perform aspects of the described functions using special-purpose hardware.

At 805, the method may include emitting light from a set of light emitting elements of the wearable device based at least in part on a known input signal to the set of light emitting elements. The operations of 805 may be performed in accordance with examples as disclosed herein. In some examples, aspects of the operations of 805 may be performed by a light emitting component 625 as described with reference to FIG. 6.

At 810, the method may include measuring, at a set of photodetectors of the wearable device, an output signal, wherein the output signal is generated by passing the light emitted from the set of light emitting elements into a material in contact with the wearable device. The operations of 810 may be performed in accordance with examples as disclosed herein. In some examples, aspects of the operations of 810 may be performed by a photodetector component 630 as described with reference to FIG. 6.

At 815, the method may include determining a hardware noise component of the output signal based at least in part on a known environmental noise component of the output signal and a comparison of the known input signal to the output signal. The operations of 815 may be performed in accordance with examples as disclosed herein. In some examples, aspects of the operations of 815 may be performed by a hardware noise component 635 as described with reference to FIG. 6.

At 820, the method may include storing the hardware noise component. The operations of 820 may be performed in accordance with examples as disclosed herein. In some examples, aspects of the operations of 820 may be performed by a memory component 640 as described with reference to FIG. 6.

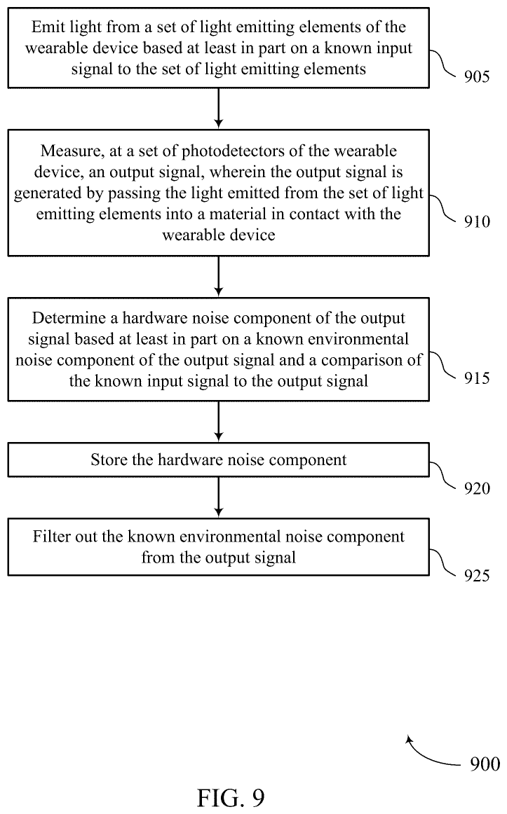

FIG. 9 shows a flowchart illustrating a method 900 that supports hardware noise filtering in accordance with aspects of the present disclosure. The operations of the method 900 may be implemented by a wearable device or its components as described herein. For example, the operations of the method 900 may be performed by a wearable device as described with reference to FIGS. 1 through 7. In some examples, a wearable device may execute a set of instructions to control the functional elements of the wearable device to perform the described functions. Additionally, or alternatively, the wearable device may perform aspects of the described functions using special-purpose hardware.

At 905, the method may include emitting light from a set of light emitting elements of the wearable device based at least in part on a known input signal to the set of light emitting elements. The operations of 905 may be performed in accordance with examples as disclosed herein. In some examples, aspects of the operations of 905 may be performed by a light emitting component 625 as described with reference to FIG. 6.

At 910, the method may include measuring, at a set of photodetectors of the wearable device, an output signal, wherein the output signal is generated by passing the light emitted from the set of light emitting elements into a material in contact with the wearable device. The operations of 910 may be performed in accordance with examples as disclosed herein. In some examples, aspects of the operations of 910 may be performed by a photodetector component 630 as described with reference to FIG. 6.

At 915, the method may include determining a hardware noise component of the output signal based at least in part on a known environmental noise component of the output signal and a comparison of the known input signal to the output signal. The operations of 915 may be performed in accordance with examples as disclosed herein. In some examples, aspects of the operations of 915 may be performed by a hardware noise component 635 as described with reference to FIG. 6.

At 920, the method may include storing the hardware noise component. The operations of 920 may be performed in accordance with examples as disclosed herein. In some examples, aspects of the operations of 920 may be performed by a memory component 640 as described with reference to FIG. 6.

At 925, the method may include filtering out the known environmental noise component from the output signal. The operations of 925 may be performed in accordance with examples as disclosed herein. In some examples, aspects of the operations of 925 may be performed by an environmental noise component 645 as described with reference to FIG. 6.

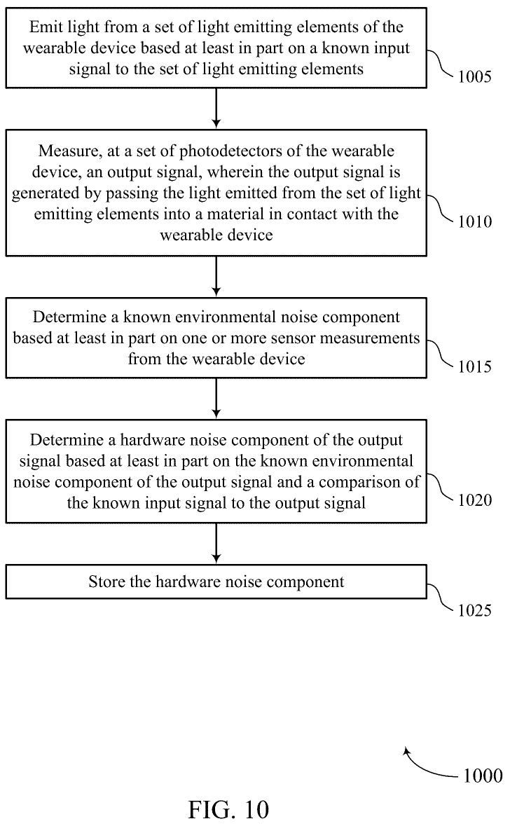

FIG. 10 shows a flowchart illustrating a method 1000 that supports hardware noise filtering in accordance with aspects of the present disclosure. The operations of the method 1000 may be implemented by a wearable device or its components as described herein. For example, the operations of the method 1000 may be performed by a wearable device as described with reference to FIGS. 1 through 7. In some examples, a wearable device may execute a set of instructions to control the functional elements of the wearable device to perform the described functions. Additionally, or alternatively, the wearable device may perform aspects of the described functions using special-purpose hardware.

At 1005, the method may include emitting light from a set of light emitting elements of the wearable device based at least in part on a known input signal to the set of light emitting elements. The operations of 1005 may be performed in accordance with examples as disclosed herein. In some examples, aspects of the operations of 1005 may be performed by a light emitting component 625 as described with reference to FIG. 6.

At 1010, the method may include measuring, at a set of photodetectors of the wearable device, an output signal, wherein the output signal is generated by passing the light emitted from the set of light emitting elements into a material in contact with the wearable device. The operations of 1010 may be performed in accordance with examples as disclosed herein. In some examples, aspects of the operations of 1010 may be performed by a photodetector component 630 as described with reference to FIG. 6.

At 1015, the method may include determining a known environmental noise component based at least in part on one or more sensor measurements from the wearable device. The operations of 1015 may be performed in accordance with examples as disclosed herein. In some examples, aspects of the operations of 1015 may be performed by an environmental noise component 645 as described with reference to FIG. 6.

At 1020, the method may include determining a hardware noise component of the output signal based at least in part on the known environmental noise component of the output signal and a comparison of the known input signal to the output signal. The operations of 1020 may be performed in accordance with examples as disclosed herein. In some examples, aspects of the operations of 1020 may be performed by a hardware noise component 635 as described with reference to FIG. 6.

At 1025, the method may include storing the hardware noise component. The operations of 1025 may be performed in accordance with examples as disclosed herein. In some examples, aspects of the operations of 1025 may be performed by a memory component 640 as described with reference to FIG. 6.

It should be noted that the methods described above describe possible implementations, and that the operations and the steps may be rearranged or otherwise modified and that other implementations are possible. Furthermore, aspects from two or more of the methods may be combined.

A method for hardware noise filtering for a wearable device is described. The method may include emitting light from a set of light emitting elements of the wearable device based at least in part on a known input signal to the set of light emitting elements, measuring, at a set of photodetectors of the wearable device, an output signal, wherein the output signal is generated by passing the light emitted from the set of light emitting elements into a material in contact with the wearable device, determining a hardware noise component of the output signal based at least in part on a known environmental noise component of the output signal and a comparison of the known input signal to the output signal, and storing the hardware noise component.

An apparatus for hardware noise filtering for a wearable device is described. The apparatus may include a processor, memory coupled with the processor, and instructions stored in the memory. The instructions may be executable by the processor to cause the apparatus to emit light from a set of light emitting elements of the wearable device based at least in part on a known input signal to the set of light emitting elements, measure, at a set of photodetectors of the wearable device, an output signal, wherein the output signal is generated by passing the light emitted from the set of light emitting elements into a material in contact with the wearable device, determine a hardware noise component of the output signal based at least in part on a known environmental noise component of the output signal and a comparison of the known input signal to the output signal, and store the hardware noise component.

Another apparatus for hardware noise filtering for a wearable device is described. The apparatus may include means for emitting light from a set of light emitting elements of the wearable device based at least in part on a known input signal to the set of light emitting elements, means for measuring, at a set of photodetectors of the wearable device, an output signal, wherein the output signal is generated by passing the light emitted from the set of light emitting elements into a material in contact with the wearable device, means for determining a hardware noise component of the output signal based at least in part on a known environmental noise component of the output signal and a comparison of the known input signal to the output signal, and means for storing the hardware noise component.

A non-transitory computer-readable medium storing code for hardware noise filtering for a wearable device is described. The code may include instructions executable by a processor to emit light from a set of light emitting elements of the wearable device based at least in part on a known input signal to the set of light emitting elements, measure, at a set of photodetectors of the wearable device, an output signal, wherein the output signal is generated by passing the light emitted from the set of light emitting elements into a material in contact with the wearable device, determine a hardware noise component of the output signal based at least in part on a known environmental noise component of the output signal and a comparison of the known input signal to the output signal, and store the hardware noise component.

In some examples of the method, apparatuses, and non-transitory computer-readable medium described herein, determining the hardware noise component may include operations, features, means, or instructions for filtering out the known environmental noise component from the output signal.

In some examples of the method, apparatuses, and non-transitory computer-readable medium described herein, the known environmental noise component comprises ambient light noise, motion artifact noise, physiological phenomenon noise, or a combination thereof.

In some examples of the method, apparatuses, and non-transitory computer-readable medium described herein, a physiological phenomenon corresponding to the physiological phenomenon noise comprises a heart rate, an oxygen saturation level, a blood pressure, or a combination thereof.

Some examples of the method, apparatuses, and non-transitory computer-readable medium described herein may further include operations, features, means, or instructions for determining the known environmental noise component based at least in part on one or more sensor measurements from the wearable device.

Some examples of the method, apparatuses, and non-transitory computer-readable medium described herein may further include operations, features, means, or instructions for determining the known environmental noise component based at least in part on an environmentally controlled calibration setup, wherein the material in contact with the wearable device comprises an artificial material configured to mimic an optical property of human tissue.

Some examples of the method, apparatuses, and non-transitory computer-readable medium described herein may further include operations, features, means, or instructions for activating, after storing the hardware noise component, the wearable device to collect a plurality of measurements of a physiological phenomenon of a wearer of the wearable device and filtering out the hardware noise component from the plurality of measurements.

Some examples of the method, apparatuses, and non-transitory computer-readable medium described herein may further include operations, features, means, or instructions for performing a plurality of measurements using the set of light emitting elements and the set of photodetectors, wherein each measurement of the plurality of measurements corresponds to a different measurement parameter configuration and determining one or more hardware noise values for each measurement parameter configuration.

Some examples of the method, apparatuses, and non-transitory computer-readable medium described herein may further include operations, features, means, or instructions for sorting the one or more hardware noise values for each measurement configuration to obtain a measurement parameter configuration range satisfying a threshold measurement accuracy value and storing the measurement parameter configuration range.

In some examples of the method, apparatuses, and non-transitory computer-readable medium described herein, the output signal corresponds to a plurality of signal paths associated with the set of light emitting elements and the set of photodetectors.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for hardware noise filtering for a wearable device, comprising:
emitting light from a set of light emitting elements of the wearable device based at least in part on a known input signal to the set of light emitting elements;
measuring, at a set of photodetectors of the wearable device, an output signal, wherein the output signal is generated by passing the light emitted from the set of light emitting elements into a material in contact with the wearable device;
determining a hardware noise component of the output signal based at least in part on a known environmental noise component of the output signal and a comparison of the known input signal to the output signal; and
storing the hardware noise component.

2. The method of claim 1, wherein determining the hardware noise component comprises:
filtering out the known environmental noise component from the output signal.

3. The method of claim 1, wherein the known environmental noise component comprises ambient light noise, motion artifact noise, physiological phenomenon noise, or a combination thereof.

4. The method of claim 3, wherein a physiological phenomenon corresponding to the physiological phenomenon noise comprises a heart rate, an oxygen saturation level, a blood pressure, or a combination thereof.

5. The method of claim 1, further comprising:
determining the known environmental noise component based at least in part on one or more sensor measurements from the wearable device.

6. The method of claim 1, further comprising:
determining the known environmental noise component based at least in part on an environmentally controlled calibration setup, wherein the material in contact with the wearable device comprises an artificial material configured to mimic an optical property of human tissue.

7. The method of claim 1, further comprising:
activating, after storing the hardware noise component, the wearable device to collect a plurality of measurements of a physiological phenomenon of a wearer of the wearable device; and
filtering out the hardware noise component from the plurality of measurements.

8. The method of claim 1, further comprising:
performing a plurality of measurements using the set of light emitting elements and the set of photodetectors, wherein each measurement of the plurality of measurements corresponds to a different measurement parameter configuration; and
determining one or more hardware noise values for each measurement parameter configuration.

9. The method of claim 8, further comprising:
sorting the one or more hardware noise values for each measurement configuration to obtain a measurement parameter configuration range satisfying a threshold measurement accuracy value; and
storing the measurement parameter configuration range.

10. The method of claim 1, wherein the output signal corresponds to a plurality of signal paths associated with the set of light emitting elements and the set of photo detectors.

11. An apparatus for hardware noise filtering for a wearable device, comprising:
a processor;
memory coupled with the processor; and
instructions stored in the memory and executable by the processor to cause the apparatus to:
emit light from a set of light emitting elements of the wearable device based at least in part on a known input signal to the set of light emitting elements;
measure, at a set of photodetectors of the wearable device, an output signal, wherein the output signal is generated by passing the light emitted from the set of light emitting elements into a material in contact with the wearable device;
determine a hardware noise component of the output signal based at least in part on a known environmental noise component of the output signal and a comparison of the known input signal to the output signal; and
store the hardware noise component.

12. The apparatus of claim 11, wherein the instructions to determine the hardware noise component are executable by the processor to cause the apparatus to:
filter out the known environmental noise component from the output signal.

13. The apparatus of claim 11, wherein the known environmental noise component comprises ambient light noise, motion artifact noise, physiological phenomenon noise, or a combination thereof.

14. The apparatus of claim 13, wherein a physiological phenomenon corresponding to the physiological phenomenon noise comprises a heart rate, an oxygen saturation level, a blood pressure, or a combination thereof.

15. The apparatus of claim 11, wherein the instructions are further executable by the processor to cause the apparatus to:
determine the known environmental noise component based at least in part on one or more sensor measurements from the wearable device.

16. The apparatus of claim 11, wherein the instructions are further executable by the processor to cause the apparatus to:
determine the known environmental noise component based at least in part on an environmentally controlled calibration setup, wherein the material in contact with the wearable device comprises an artificial material configured to mimic an optical property of human tissue.

17. The apparatus of claim 11, wherein the instructions are further executable by the processor to cause the apparatus to:
activate, after storing the hardware noise component, the wearable device to collect a plurality of measurements of a physiological phenomenon of a wearer of the wearable device; and
filter out the hardware noise component from the plurality of measurements.

18. The apparatus of claim 11, wherein the instructions are further executable by the processor to cause the apparatus to:
perform a plurality of measurements using the set of light emitting elements and the set of photodetectors, wherein each measurement of the plurality of measurements corresponds to a different measurement parameter configuration; and
determine one or more hardware noise values for each measurement parameter configuration.

19. The apparatus of claim 18, wherein the instructions are further executable by the processor to cause the apparatus to:
sort the one or more hardware noise values for each measurement configuration to obtain a measurement parameter configuration range satisfying a threshold measurement accuracy value; and
store the measurement parameter configuration range.

20. A non-transitory computer-readable medium storing code for hardware noise filtering for a wearable device, the code comprising instructions executable by a processor to:
emit light from a set of light emitting elements of the wearable device based at least in part on a known input signal to the set of light emitting elements;
measure, at a set of photodetectors of the wearable device, an output signal, wherein the output signal is generated by passing the light emitted from the set of light emitting elements into a material in contact with the wearable device;
determine a hardware noise component of the output signal based at least in part on a known environmental noise component of the output signal and a comparison of the known input signal to the output signal; and
store the hardware noise component.
